(19) United States
(12) Plant Patent Application Publication
(10) Pub. No.: US 2015/0319903 P1
(43) Pub. Date: Nov. 5, 2015

(54) Calceolaria plant named 'KLECH13020'

(71) Applicant: Klemm+Sohn GmbH & Co. KG, Stuttgart (DE)

(72) Inventors: Nils Klemm, Stuttgart (DE); Katinka Wilde, Stuttgart (DE)

(73) Assignee: Klemm+Sohn GmbH & Co. KG, Stuttgart (DE)

(21) Appl. No.: 14/120,149

(22) Filed: Apr. 30, 2014

(51) Int. Cl. A01H 5/00

(52) U.S. Cl. PLT/263.1

(57) ABSTRACT

A new Calceolaria plant particularly distinguished by having a red-black flower color, a red-black upper lip (hood) with a yellow border, and a stretching plant habit, is disclosed.

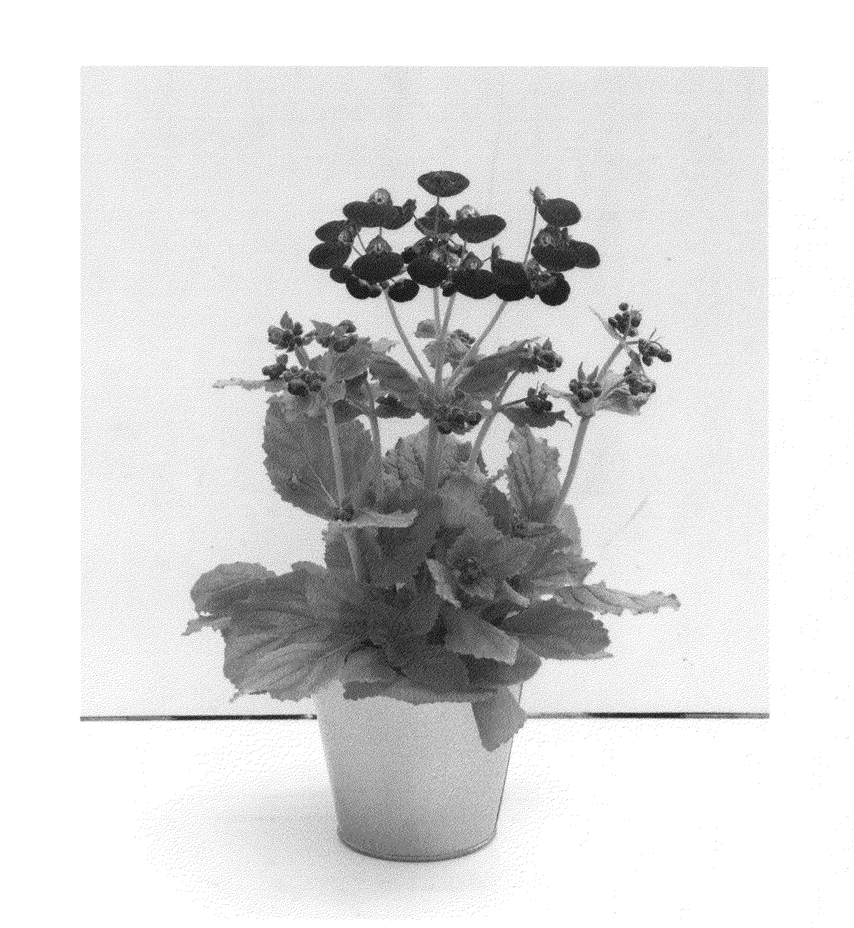

### GENUS AND SPECIES

Calceolaria hybrida

### VARIETY DENOMINATION

‘KLECH13020’

### BACKGROUND OF THE NEW PLANT

The present invention comprises a new and distinct variety of Calceolaria, botanically known as Calceolaria hybrida and hereinafter referred to by the variety name ‘KLECH13020’. ‘KLECH13020’ is a selection from a cross-pollination of the proprietary female Calceolaria line ‘CH-2008-0199’ (unpatented) and the proprietary male Calceolaria line ‘CH-2008-0198’ (unpatented).

In June of 2008 the proprietary female Calceolaria line ‘CH-2008-0199’ and the proprietary male Calceolaria line ‘CH-2008-0198’ were cross pollinated in Stuttgart, Germany and seeds were obtained. The seeds were sown in the winter of early 2009 and grown in a greenhouse after crop specific cultivation selection was done. A plant line was selected in Stuttgart, Germany in May of 2009 and named ‘KLECH13020’. First cuttings of ‘KLECH13020’ were taken in June 2009 in Stuttgart, Germany to build up an elite stock and has been propagated six times by cuttings. ‘KLECH13020’ was found to reproduce true to type in successive generations of asexual propagation via cuttings in Stuttgart, Germany.

### CROSS REFERENCE TO RELATED APPLICATIONS

Plant Breeder's Rights for ‘KLECH13020’ were applied for in Canada on Feb. 3, 2014 (14-8193). ‘KLECH13020’ has not been made publicly available or sold more than one year prior to the filing date of this application.

### SUMMARY

The following are the most outstanding and distinguishing characteristics of this new variety when grown under normal horticultural practices in Stuttgart, Germany.

1. Red-black flower color;

2. A red-black upper lip (hood) with a yellow border; and

3. A stretching plant habit.

### DESCRIPTION OF THE PHOTOGRAPH

This new Calceolaria plant is illustrated by the accompanying photograph which shows an overall view of the plant. The plant is about 3 months-old grown indoors in Stuttgart, Germany in April 2013. The colors shown are as true as can be reasonably obtained by conventional photographic procedures.

### DESCRIPTION OF THE NEW VARIETY

The following detailed descriptions set forth the distinctive characteristics of ‘KLECH13020’. The data which define these characteristics were collected from asexual reproductions carried out in Stuttgart, Germany. Data was collected on three-month-old plants grown indoors in Stuttgart, Germany under greenhouse conditions in April of 2013. Color references are to The R.H.S. Colour Chart of The Royal Horticultural Society of London (R.H.S.), 5^{th} edition (2007).
Classification:
   Family.—Scrophulariaceae or Calceolariaceae.
   Species.—Calceolaria hybrid.
   Common name.—Lady's purse, slipper flower, pocketbook flower.
   Variety.—‘KLECH13020’.
Parentage:
   Female.—Proprietary Calceolaria line ‘CH-2008-0199’.
   Male.—Proprietary Calceolaria line ‘CH-2008-0198’.
Plant:
   Form.—Potted plant.
   Habit.—Upright.
   Plant height.—Approximately 27.0 cm.
   Plant width.—Approximately 33.0 cm.
   Flowering habit.—Continuous, but intermittent in summertime.
   Time to produce a finished flowering plant.—10 to 11 weeks (in 12 cm pot).
   Propagation type.—Cuttings.
   Time to initiate roots.—5 to 6 weeks.
   Root description.—Brown.
Stems:
   Quantity per plant.—5 to 8.
   Color.—RHS 187A, then with maturity getting lighter and greener to RHS 142B.
   Length.—Approximately 6.0 cm.
   Diameter.—Approximately 0.5 cm.
   Internode length.—Approximately 2.5 cm.
   Texture.—Less pubescent, hairs are very small and soft, not sticky.
   Anthocyanin.—Present, RHS 187A.
Leaves:
   Arrangement.—Decussate.
   Length.—2.0 cm (small, younger leaves); 9.5 cm (bigger, old leaves).
   Width.—1.0 cm (small, younger leaves); 7.5 cm (bigger, old leaves).
   Shape.—Rhombical.
   Apex.—Obtuse.
   Base.—Attenuate.
   Margin.—Crenate.
   Texture.—Leathery with very small hairs.
   Color.—Immature leaf: Upper surface: RHS 137B to RHS 137A Lower surface: RHS 137D Mature leaf: Upper surface: RHS 137B to RHS 137A Lower surface: RHS 137D.
   Venation pattern.—reticulate.
   Venation color.—Upper surface: RHS 137D Lower surface: RHS 137D.
   Petiole.—Length: Approximately 1.0 cm Diameter: 0.5 cm to 1.5 cm Color: RHS 137D with anthocyanin RHS 187A Texture: Pubescent, but hairs are very small and not sticky.
Flowers:
   General.—Zygomorphic with a four-parted calyx, with the lobes free to the base, and a two-lipped sympetalous corolla.
   Flowering season.—Without artificial light, from May to June and August to October.
   Lastingness of individual flowers on the plant.—About 3 weeks.
   Fragrance.—Absent.
Bud:
   Shape.—Truncate hemisphere.
   Height.—About 0.4 cm.
   Width.—0.6 cm to 1.5 cm.
   Color.—RHS 187B.
   Texture.—Velvety, not pubescent.
Corolla:
   General.—The corolla is two-lipped and composed of two pouches, the upper lip and the lower lip.
   Upper lip.—Hood: Present, RHS 187B with light yellow border RHS 4D Length: Approximately 1.2 cm Width: Approximately 0.3 cm Texture: Velvety, not pubescent Color: Immature: RHS 187A Mature: RHS 187A changing to lighter RHS 187B with maturity Shape: Hemispherical Apex: Elliptical Base: Fused with the lower lip more flattened Margin: Entire.
   Lower lip.—Length: Approximately 2.3 cm Width: Approximately 2.6 cm Texture: Velvety, not pubescent Color: Immature: RHS 187A Mature: RHS 187B Shape: Hemispherical Apex: Elliptical Base: Fused with upper lip more flattened Margin: Entire Oil gland: Absent.
Calyx: Four-parted calyx; all calyx lobes showing similar size.
   Sepals.—Upper Sepal: Length: Approximately 0.5 cm Width: Approximately 0.6 cm Color: Upper surface: RHS 137C Lower surface: RHS 137D with anthocyanin RHS 187A Texture: Leathery Shape: Ovate (egg shape) with acuminate sepal tip Apex: Acuminate Base: Fused with three other sepals Margin: Entire Lateral sepals: Length: Approximately 0.5 cm Width: Approximately 0.6 cm Color: Upper surface: RHS 137C Lower surface: RHS 137D with anthocyanin RHS 187A Texture: Leathery Shape: Ovate with acuminate sepal tip Apex: Acuminate Base: Fused with other sepals Margin: Entire.
   Pedicel.—Length: Approximately 1.5 cm Diameter: Approximately 0.1 cm Color: RHS 137D with anthocyanin RHS 187C Texture: Smooth.
   Peduncle.—Length: 3.0 cm to 10.0 cm Diameter: Approximately 0.3 cm Color: RHS 144D Texture: Pubescent, sticky.
Reproductive organs:
   Stamens.—Quantity per flower: 2 Anther shape: Elliptic (Theca) Anther length: 0.2 cm (length of connective—Theca approximately 0.2 cm) Anther color: RHS 4D Pollen amount: Scarce Pollen color: RHS 4D.
   Pistils.—Quantity per flower: 1 Pistil length: Approximately 0.2 cm Style length: Approximately 0.3 cm Style color: RHS 187A Stigma shape: Tube-like Stigma color: RHS 4D Ovary color: RHS 187B.
Fruit and seed set: After pollination, cark capsulates are developed. They are ovate-shaped, seeds are brown-black, oblong to elliptic.
Disease and insect resistance: No particular disease or insect resistance observed.

### COMPARISON WITH PARENTAL LINES AND KNOWN VARIETY

‘KLECH13020’ is a distinct variety of Calceolaria. ‘KLECH13020’ is distinguished from its parents as described in Table 1:

TABLE 1
Comparison with Parental Lines

| Trait or | Variety | Female parent | Male Parent |
|---|---|---|---|
| characteristic | ‘KLECH13020’ | ‘CH-2008-0199’ | ‘CH-2008-0198’ |
| Habit | Stretching | Stretching | Not stretching |
| Flower color | Red-black | Red-black | Dark red |
| Flower size | Medium | Small | Medium |

‘KLECH13020’ is most similar to the commercial calceolaria plant named ‘Calynopsis Red’ (unpatented). Differences between the two varieties are described in Table 2:

TABLE 2
Comparison with Similar Variety

| | Variety | Commercial line: |
|---|---|---|
| Trait or characteristic | ‘KLECH13020’ | ‘Calynopsis Red’ |
| Flower color | Red-black | Red |
| Plant habit | Stretching | Not stretching |
| Upper lip (hood) | Red-black/light yellow border | Yellow |

We claim:

1. A new and distinct variety of Calceolaria plant named ‘KLECH13020’ as shown and described herein.
